(19) United States
(12) Patent Application Publication
(10) Pub. No.: US 2022/0240792 A1
(43) Pub. Date: Aug. 4, 2022

(54) Vessel Measurements

(71) Applicant: Foundry Innovation & Research 1, Ltd., Dublin (IE)

(72) Inventors: Friedrich Wetterling, Dublin (IE); Conor M. Hanley, Dublin (IE); Fiachra M. Sweeney, Dublin (IE)

(21) Appl. No.: 17/622,313

(22) PCT Filed: Jun. 24, 2020
(86) PCT No.: PCT/EP2020/067713

§ 371 Date: Dec. 23, 2021

Related U.S. Application Data
(60) Provisional application No. 62/865,750, filed on Jun. 24, 2019.

(51) Int. Cl. A61B 5/02; A61B 5/024; A61B 5/00

(52) U.S. Cl. CPC A61B 5/02007; A61B 5/746; A61B 5/024

(57) ABSTRACT

Systems and methods are described relating to fluid volume sensing in the inferior vena cava (IVC) to obtain data from which information on fluid status, congestion and cardiac output may be derived.

### FIELD

The present disclosure generally relates to the field of vascular monitoring. In particular, the present disclosure is directed to wireless vascular monitoring implants, systems, methods, and software. More specifically, embodiments disclosed herein relate to fluid volume sensing in the venae cavae (Inferior and superior venae cavae) to obtain data from which information on fluid status, congestion and cardiac output may be derived.

### BACKGROUND

Heart failure, also often referred to, as congestive heart failure, occurs when the myocardium cannot efficiently provide oxygenated blood to the vascular system. A variety of pathophysiological conditions, such as myocardial damage, diabetes mellitus, and hypertension gradually disrupt organ function and autoregulation mechanisms, leaving the heart unable to properly fill with blood and eject it into the vasculature. In parallel, heart failure can interact unfavourably with a series of complications such as heart valve problems, arrhythmias, liver damage and renal damage or failure.

Others have attempted to develop vascular monitoring devices and techniques, including those directed at monitoring vessel arterial or venous pressure or vessel lumen dimensions.

However, many such existing systems are catheter based (not wireless) and thus can only be utilized in a clinical setting for limited periods of time, and may carry risks associated with extended catheterization. For a wireless solution, the complexity of deployment, fixation and the interrelationship of those factors with detection and communication have led to, at best, inconsistent results with such previously developed devices and techniques.

Existing wireless systems focus on pressure measurements, which in the IVC can be less responsive to patient fluid state than IVC dimensional measurements. However, systems designed to measure vessel dimensions also have a number of drawbacks with respect to monitoring in the IVC. Electrical impedance-based systems require electrodes that are specifically placed in opposition across the width of the vessel. Such devices present special difficulties when attempting to monitor IVC dimensions due to the fact that the IVC does not expand and contract symmetrically as do most other vessels where monitoring may be desired. Precise positioning of such position-dependent sensors is a problem that has not yet been adequately addressed. IVC monitoring presents a further challenge arising from the physiology of the IVC. The IVC wall is relatively compliant compared to other vessels and thus can be more easily distorted by forces applied by implants to maintain their position within the vessel. Thus, devices that may perform satisfactorily in other vessels may not necessarily be capable of precise monitoring in the IVC due to distortions created by the force of the implant acting on the IVC wall. As such, new developments in this field are desirable in order to provide doctors and patients with reliable and affordable wireless vascular monitoring implementation, particularly in the critical area of heart failure monitoring.

### SUMMARY

Embodiments of the present disclosure provide a system for determining fluid status in a blood vessel comprising:
a sensor configured to obtain a measurement from the vessel;
a processor configured to:
   derive a measure of cardiac collapse of the vessel from the measurement;
   derive a measure of respiratory collapse of the vessel from the measurement;
   calculate a ratio of cardiac to respiratory collapse such that the calculated ratio provides an indication of the fluid status in the vessel.

This is advantageous as it provides for obtaining an indication of a fluid status independent of inter- and intra-individual variations of the quantitative measurement itself. For example, wherein the sensor obtains a signal type measurement, it provides that features in the signal may be used to derive a fluid status rather than an absolute physical measure of the vessel such as pressure, or volume Two aspects of the same absolute physical effect, i.e. collapse resulting from cardiac action and collapse resulting from respiratory action, are derived from a measurement in order to compute a ratio of vessel change depending on the cardiac and respiratory activity. In doing so, the measurement is normalised, thus removing the impact of error sources such as the effect of in-growth as well as inter- and intra-individual variations, patient position differences or differences in intra-abdominal pressures.

The sensor of the system may be deployed in the blood vessel. This is advantageous as, once deployed, the sensor may be used to provide simple, accurate, non-invasive, measurements as required. The need for repeated invasive measurements to be taken from a patient is obviated.

The sensor of the system may be applied to the skin of a patient. This is advantageous as, once again, the sensor may be used to provide measurements as required. Furthermore, applying the sensor to the skin of a patient provides a straightforward and non-invasive manner to obtain patient data.

The measurement may be a pressure measurement. Pressure measurements of a vessel provide a key indicator of fluid status. This pressure measurement may be obtained from an implantable within the vessel or externally via an external pressure measurement device.

The measurement may be in the form of an MM image. Such images provide important visual indications of the physical state of a vessel and its fluid status. Such images also provide important visual clues of potential risks to a patient.

The measurement may be obtained via ultrasound (external, internal, intravascular, and or other access to capture image region of interest). This common tool may be used to obtain the raw measurement traces that can then be analysed to provide information about the patient's fluid status.

The measurement may be a pulse oximetry measurement. This is advantageous as the pulse oximetry provides information as to the blood's oxygen levels.

The above described measurements may be used to obtain a ratio of cardiac to respiratory collapse such that the calculated ratio provides an indication of the fluid status in the vessel.

The measurement may be a temporal trace recording. Furthermore, the temporal trace recording may be of vascular modulation from the vessel. This is advantageous as it provides for continuous or ongoing measurement and monitoring of fluid status. This is important as it provides for changes in fluid status to be visualised over time. This further provides that predictions of future fluid status may be obtained. This provides that pre-emptive treatment may be assigned to a patient before their current condition deteriorates.

Further provided is a method of determining fluid status in a blood vessel comprising:
obtaining a measurement from the vessel via a sensor;
deriving a measure of cardiac collapse from the measurement;
deriving a measure of respiratory collapse from the measurement;
calculating a ratio of cardiac to respiratory collapse such that the calculated ratio provides an indication of the fluid status in the vessel.

The method may further comprise adjusting the volume of fluid in the vessel based on the indication of the fluid status. In this manner, the method provides that a patient's fluid status may be regulated based on the provided measurements. Adjusting the volume of fluid in the vessel may comprise one or a combination of drug intake, dialysis, ultrafiltration, blood pumping. The obtained measurements can provide indications as to the most appropriate treatment schedule for a given patient.

Further provided is a system for determining fluid status in a blood vessel comprising:
a resilient sensor, deployed in the blood vessel, configured to obtain a measurement from the vessel, the sensor being compressible between a maximally dimensioned size s1, and a minimally dimensioned size s2;
a processor configured to:
   obtain a measurement, m1, of the change in sensor dimensions after deployment in the vessel, m1 being a value between and including s1 and s2;
   obtain from m1, a value of a radial force, r1 exerted by the sensor on the vessel after deployment in the vessel;
   calculate a ratio of the change in a vessel dimension resulting from r1 after deployment of the sensor in the vessel with respect to a known vessel dimension, Ao, prior to deployment of the sensor in the vessel, wherein the ratio provides an indication of the fluid status in the vessel.

This is advantageous as it provides that fluid status may be derived based on the force exerted by the spring due to its compression and extension within a blood vessel after deployment in the vessel.

The system wherein, m1 is a measurement of maximum sensor dimensions after deployment in the vessel, m1 being a value between and including s1 and s2; and the processor may be further configured to obtain a second measurement, m2, of minimum sensor dimensions after deployment in the vessel, m2 being a value between and including s1 and s2 and obtain from m1 and m2, a value of the radial force, r1 exerted by the sensor on the vessel after deployment in the vessel.

Obtaining two measures (at minimum and at maximum) of the same absolute physical measure allows for a calculation of a ratio of vessel change depending on the applied force. As a result, it is possible to assess the ability of the vessel to expand and hence its capacity for containing more fluid. This provides that a vessels positon on the pressure volume curve may be ascertained and thus provide an indication of the current fluid status of the vessel and patient.

The processor may be further configured to calculate a ratio of the change of m1 with respect to a known maximum vessel dimension mₙₐₜᵢᵥₑ₁ to provide a MAXCHANGE value and a ratio of the change of m2 with respect to a known minimum vessel dimension mₙₐₜᵢᵥₑ₂ to provide a MINCHANGE value.

A full fluid status (hypervolemia) may be indicated by a MAXCHANGE value being less than a MINCHANGE value by a factor of F1, wherein F1 is about 10. This is advantageous as a comparison of the values obtained allows for an indication of a full fluid status to be ascertained.

A normal fluid status (euvolemia) is indicated by a MAXCHANGE value being higher or lower than a MINCHANGE value by a factor of F2, wherein F2 is about 2. This is advantageous as a comparison of the values obtained allows for an indication of a normal fluid status to be ascertained.

A low fluid status (hypovolemia) is indicated by a MAXCHANGE value being higher than a MINCHANGE value by a factor of F3, wherein F3 is about 1.2 to 1.5. This is advantageous as a comparison of the values obtained allows for an indication of a low fluid status to be ascertained.

The processor may be further configured to provide a notification based on the indicated fluid status the blood vessel. The indicated fluid status may be computed by an algorithm incorporating a number of features from the signal and previous signals obtained. This is advantageous as it provides an automatic presentation of information regarding the fluid status without the need for further analysis or computation.

The processor may be further configured to provide a notification indicating an action for adjusting the fluid status in the blood vessel. This is advantageous as it provides that remedial action may be automatically suggested in the event that a non-normal fluid status is indicated.

The action may comprise one or more of a drug treatment change or a medical treatment change. The obtained measurements can provide indications as to the most appropriate treatment to adjust fluid status for a given patient.

Further provided is a method for determining fluid status in a blood vessel comprising:
obtaining a vessel dimension prior to deployment of a sensor in the vessel;
deploying the sensor in the vessel, the sensor configured to obtain a measurement from the vessel, the sensor being compressible between a maximally dimensioned size s1, and a minimally dimensioned size s2;
obtaining a measurement, m1, of the change in sensor dimensions after deployment in the vessel, m1 being a value between and including s1 and s2;
obtaining from m1, a value of a radial force, r1 exerted by the sensor on the vessel after deployment in the vessel;

calculating a ratio of the change in a vessel dimension resulting from r1 after deployment of the sensor in the vessel, with respect to the obtained vessel dimension prior to deployment of the sensor in the vessel, wherein the ratio provides an indication of the fluid status in the vessel.

Further provided is a system for determining congestion in a blood vessel comprising:
a sensor in the vessel, the sensor configured to obtain a first signal indicating a first area measurement, a1, of the vessel prior to patient manoeuvre and a second signal indicating a second area measurements, a2, of the vessel after a patient manoeuvre;
a processor configured to determine the congestion in a blood vessel of the vessel based on the first and second signals.

This is advantageous as it provides that fluid status may be evaluated without the requirement for complex invasive procedures and it provides a method to subject the patient to a controlled manoeuvre to exert a controlled perturbation on the vascular system and monitor the resulting physiological change which in turn provides an indication of the patients fluid status.

The processor may be further configured to determine fluid status based on an identified signal shape derived from the first and second area measurements. The signal shape may be a square wave shape. This is advantageous as a signal obtained from the first and second area measurements provides a readily identifiable shape which is an indicator of fluid status.

The processor may be further configured to provide a notification of fluid status. This is advantageous as it provides an automatic presentation of information regarding congestion without the need for further analysis or computation.

The processor may be further configured to provide a notification indicating an action for reducing congestion. This is advantageous as it provides that remedial action may be automatically suggested in the event that congestion is indicated.

The action may comprise one or more of a drug treatment change or a medical treatment change. The obtained measurements can provide indications as to the most appropriate treatment to alleviate congestion for a given patient.

The medical treatment schedule may comprise at least of a diuretic or vasodilation schedule, a modification to a medical device such as vascular pump, drug pump, dialysis or auto-filtration machine, pacing device or extracorporeal membrane oxygenation (ECMO) machine.

Further provided is method for determining congestion comprising:
obtaining a first signal indicating a first area measurement, a1, from a sensor in a blood vessel prior to performing a patient manoeuvre;
performing the patient manoeuvre;
obtaining a second signal indicating a second area measurement, a2, from the sensor in the blood vessel after performing the patient manoeuvre;
determining the congestion in a blood vessel of the vessel based on the first and second signals.

The patient manoeuvre may be a Valsalva or sniff type manoeuvre. This is advantageous as it does not require complicated actions to be performed by the patient in order for the required measurements to be obtained. The Valsalva manoeuvre may involve the use of a device for the patient to generate a controlled level of internal pressure.

Further provided is a system for determining cardiac output, O_{c} comprising:
a sensor deployed in the inferior vena cava, IVC, the sensor configured to obtain a first area measurement, Area1, of the IVC at a time t1;
the sensor configured to obtain a second area measurement, Area2, of the IVC at a time t2;
a processor configured to determine the cardiac output based on area changes of the IVC derived from the first and second area measurements.

The system may be further configured to derive a heart rate from an analysis of the area changes of the IVC.

This is advantageous as it provides that cardiac output may be indicated without the requirement for complex invasive procedures. The processor may be configured to determine the cardiac output as the cardiac output is proportional to the change in area of the IVC.

The processor may be further configured to provide a notification of the cardiac output. This is advantageous as it provides an automatic presentation of information regarding cardiac output without the need for further analysis or computation.

The processor may be further configured to provide a notification indicating an action for adjusting the cardiac output. This is advantageous as it provides that remedial action may be automatically suggested in the event that non-normal output is indicated.

The action may comprise one or more of a drug treatment change or a medical treatment change. The obtained measurements can provide indications as to the most appropriate treatment to adjust cardiac output for a given patient.

The medical treatment schedule may comprise at least of a diuretic or vasodilation schedule, a modification to a medical device such as vascular pump, drug pump, dialysis or auto-filtration machine, pacing device or extracorporeal membrane oxygenation (ECMO) machine.

Further provided is a method for determining cardiac output comprising:
obtaining a first area measurement, Area1, from a sensor deployed in the inferior vena cava, IVC, at a time t1;
obtaining a second area measurement, Area2, from a sensor deployed in the IVC, at a time t2;
determining the cardiac output based on area changes of the IVC derived from the first and second area measurements.

The method may further comprise deriving a heart rate from an analysis of the area changes of the IVC.

### BRIEF DESCRIPTION OF THE DRAWINGS

FIG. 1a is a schematic plot of patient fluid volume versus response employing IVC diameter or area measurement (curves A1 and A2) in comparison to prior pressure-based systems (curve B) and in general relationship to IVC collapsibility index (IVC CI, curve C).

FIG. 1b is a plot of data from an in vivo fluid removal and loading experiment.

FIG. 2 shows a measurement being obtained from a patient via an embodiment of the system according to the disclosure

FIG. 3 shows a schematic example of a sensor which can be used according to the system of the disclosure

FIG. 4 shows an example of a sensor which can be used according to the system of the disclosure FIG. 5 shows a plot of absolute cross section of a sensor (in mm²) over a period of time.

FIGS. 6A and 6B are plots showing a comparison of example data from loading blood into healthy sheep (weight=70 kg) under dry, normal and wet conditions

FIGS. 7A and 7B show IVC area and pressure changes in a native vessel (top image labelled “native vessel”) and after deployment of a sensor device into the IVC (lower image labelled “acute after sensor deployment”).

FIG. 8 shows data obtained from fluid loading testing in the IVC of sheep

FIG. 9 shows data obtained from fluid loading testing in a native IVC of a heart failure patient.

FIG. 10 shows a determination of CVP-A/A0 curve experimentally for a vein.

FIGS. 11 and 12 shows a calibration procedure to acquire radial force versus sensor area data

FIG. 13 shows an adjusted model merging a radial force of sensor curve and an experimentally obtained native vessel curve for pressure and volume/area.

FIG. 13A shows a schematic of a sensor sizes in a vessel

FIG. 14 shows radial force results for an expiration area in the flat part of the CVP-A curve

FIG. 15 shows radial force results for an expiration area in the end of the flat part of the CVP-A curve

FIG. 16 shows radial force results for an expiration area in the steep part of the CVP-A curve

FIG. 17 shows a square wave response in blood pressure to a Valsalva manoeuvre

### DETAILED DESCRIPTION

Use of Area Measurements of Blood Vessels

The assignee of the present disclosure has developed a number of devices that provide fluid volume data based on direct measurement of physical dimensions of blood vessels such as the diameter or area. Examples of these devices are described, for example, in PCT/US2016/017902, filed Feb. 12, 2016, and WO2018/031714, filed Aug. 10, 2017 by the present Applicant, each of which is incorporated by reference herein in its entirety. Devices of the types described in these prior disclosures facilitate new management and treatment techniques based on regular intermittent (e.g., daily) or substantially continuous (near real-time), direct feedback on physical dimensions of blood vessels.

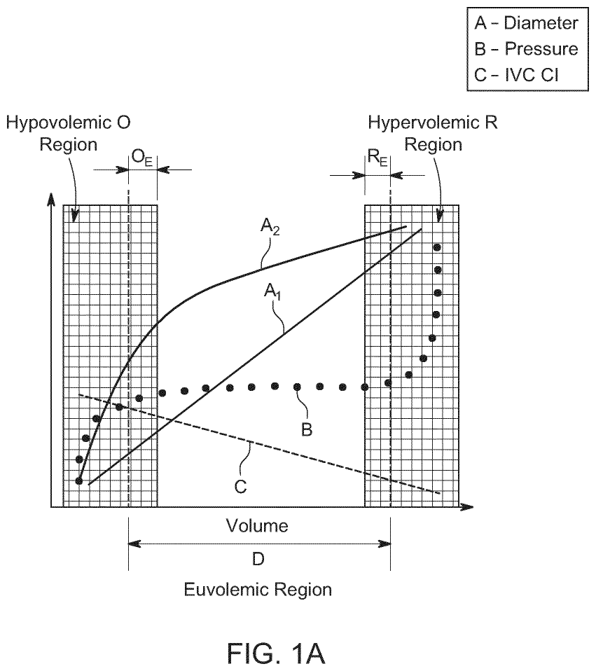

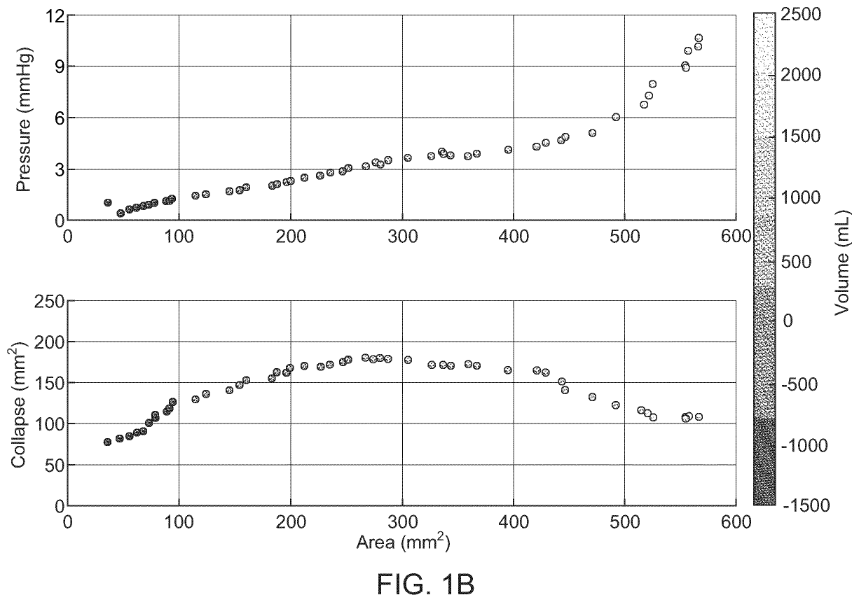

WO2018/031714 further describes some of the advantages of the information that can be derived from taking area type measurements using these devices. As can be seen in FIG. 1a (reproduced from FIG. 1 of WO2018/031714), the response of pressure-based diagnostic tools (B) over the euvolemic region (D) is relatively flat and thus provides minimal information as to exactly where patient fluid volume resides within that region. Pressure-based diagnostic tools thus tend to only indicate measureable response after the patient's fluid state has entered into the hypovolemic region (O) or the hypervolemic region (R). In contrast, a diagnostic approach based on diameter or area measurement across the respiratory and/or cardiac cycles (Ai and A₂), which correlates directly to r C volume and IVC CI (hereinafter “IVC Volume Metrics”) provides relatively consistent sensitive information on patient fluid state across the full range of states.

It is noted in WO2018/031714 that using vessel area measurement, in this example with respect to the inferior vena cava (IVC), as an indicator of patient fluid volume provides an opportunity for earlier response both as a sensitive hypovolemic warning and as an earlier hypervolemic warning. With respect to hypovolemia, when using pressure as a monitoring tool, a high pressure threshold can act as a potential sign of congestion, however when pressure is below a pressure threshold (i.e., along the flat part of curve B), it gives no information about the fluid status as the patient approaches hypovolemia. With respect to hypervolemia, vessel area measurements, for example potentially provide an earlier signal than pressure-based signals due to the fact that IVC diameter or area measurements change a relatively large amount without significant change in pressure. Hence, a threshold set on IVC diameter or area measurements can give an earlier indication of hypervolemia, in advance of a pressure-based signal. FIG. 1b is a plot of data from an in vivo fluid removal and loading experiment. It shows right atrial pressure (RAP) as a function of IVC area (top) and collapse (=Amax−Amin) as a function of IVC area (below) as an example of the response of an IVC in an in-vivo fluid removal and loading experiment.

Obtaining Area Measurements of Blood Vessels

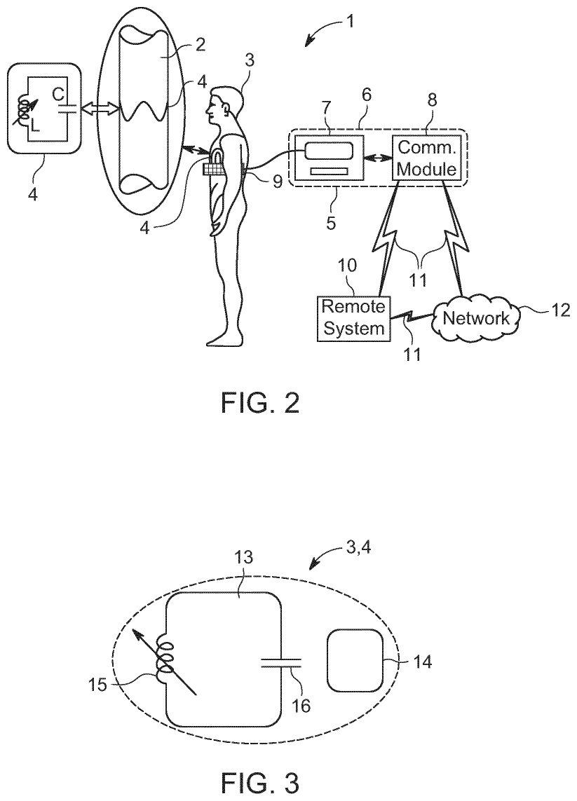

Systems and sensors for obtaining area measurements of blood vessels are described in WO2018/031714. FIG. 2 shows aspects of such a system 1 for obtaining measurements from the IVC 2 of a patient 3 utilizing a sensor 4. The system may also be utilized to obtain measurements from other vessel types.

A processor 5 may take the form of a laptop or desktop computer. The processor 5 may further be a mobile telecommunication device such as a mobile telephone or tablet. The processor may further be a wearable electronic device or sensor reader. In the case that the processor is incorporated into the sensor reader, the reader shall be capable of wirelessly transmitting and receiving the required radiofrequency pulses, filtering and processing them as required and operating the appropriate software for interpreting the results. The processor is configured with suitable software for interpretation of the sensor measurements. The sensor 4 and processor 5 may in some embodiments be further configured to communicate with control and communications modules, and one or more remote systems such as processing systems, user interface/displays, data storage, etc., communicating with the control and communications modules through one or more data links, preferably remote/wireless data links. FIG. 2 shows aspects of such systems. Such a system may include a control module 6 to communicate with and, in some embodiments, power or actuate the sensor. The processor may be comprised within the control module 6. Alternatively, the processor 5 may be as a separate device. Control module 6 may include controller 7 and communications module 8. The control module may comprise a bedside console. For patient comfort, as well as repeatability in positioning, a belt reader or antenna 9 may be worn by the patient around the waist. The antenna may serve to wirelessly transmit measurements from the sensor 4 to the processor 5. Information may be transferred 11 from the communications module 8 via Bluetooth, cellular, or local area network to a remote system 10 and/or to a network 12 for storage and/or further analysis.

The sensor 4 may take the form of an implantable device. The insertion of such devices into the circulatory system of a human or animal is well known in the art and is not described in detail here. To obtain measurements m1 and m2, the sensors are thus implanted into a blood vessel, with the first sensor at position x1 and the second sensor at position x2. Once in position and activated, the sensors are capable of obtaining modulating area measurements from the vessel via modulations in their inductance and therefore frequency. The processor obtains the measurements from the sensors by, for example, wireless link to or resonant coupling with the sensors. Once obtained by the processor, the measurements are processed and analysed as set out in further detail below to determine the dimensions of the blood vessel.

Measurements of vessel diameter or area by the sensor 4 may be made continuously over one or more respiratory cycles to determine the variations in vessel dimensions over this cycle. Further, these measurement periods may be taken continuously, at preselected periods and/or in response to a remotely provided prompt from a signal within the system or from a health care provider/patient.

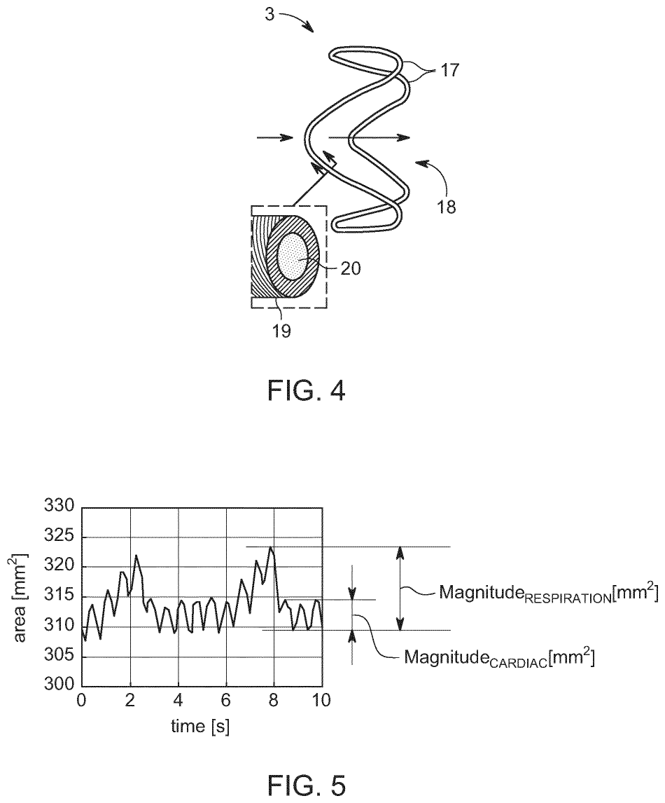

In an embodiment of the disclosure, the first sensor 4 may employ a variable inductance L-C circuit 13 for performing measuring or monitoring functions described herein, as shown schematically in FIG. 3. The sensor 4 may also include means 14 for securely anchoring the implant within the IVC. Using a variable inductor 15 and known capacitance 16, L-C circuit 13 produces a resonant frequency that varies as the inductance is varied. Changes in shape or dimension of the vessel cause a change in configuration of the variable inductors, which in turn cause changes in the resonant frequency of the circuits.

Thus, not only should the sensor be securely positioned at a monitoring position, but also, at least a variable coil/inductor portion 13 of the implant may have a predetermined compliance (resilience) selected and specifically configured to permit the inductor to move with changes in the vessel wall shape or dimension while maintaining its position with minimal distortion of the natural movement of the vessel wall. Thus, in some embodiments, the variable inductor is specifically configured to change shape and inductance in proportion to a change in the vessel shape or dimension.

Variable inductor 15 is configured to be remotely energized by an electric field delivered by one or more transmit coils within antenna module 9 positioned external to the patient. When energized, L-C circuit 13 produces a resonant frequency which is then detected by one or more receive coils of the antenna module. Because the resonant frequency is dependent upon the inductance of the variable inductor, changes in shape or dimension of the inductor caused by changes in shape or dimension of the vessel wall cause changes in the resonant frequency. The detected resonant frequency is then analysed by the processor component of the system to determine the vessel diameter or area, or changes therein. The vessel measurements obtained by the sensors are processed and analysed to determine the dimensions of the blood vessel as set out in further detail below.

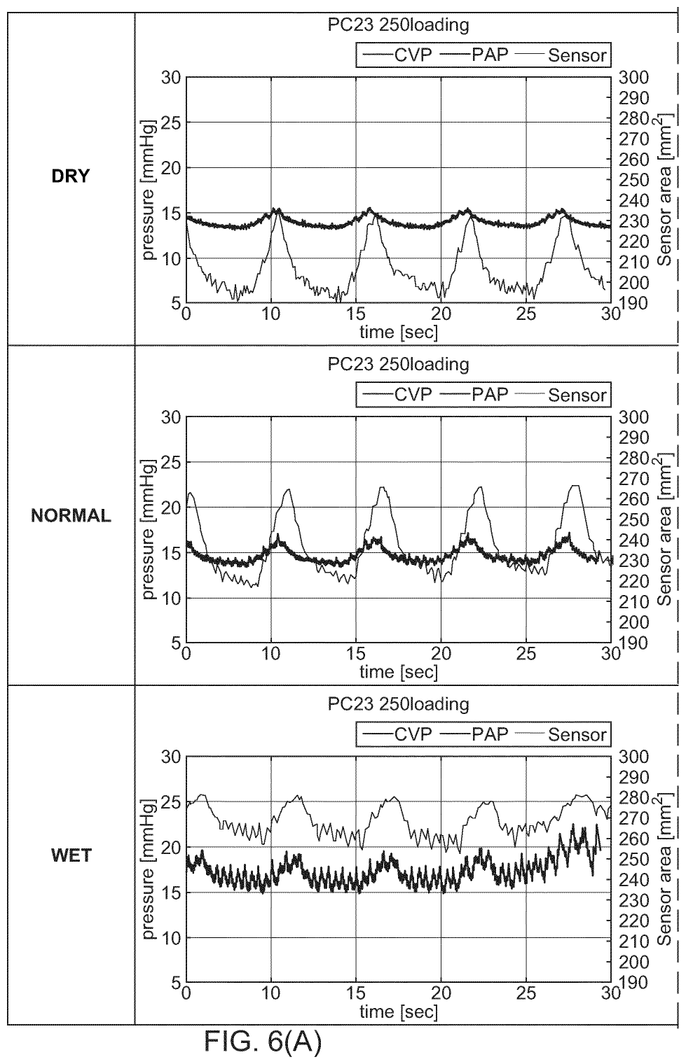

An example of a sensor 4 for use with systems and methods described herein are shown in FIG. 4 and described further below. The sensor comprises a frame with eight crowns 17. The enlarged detail in the box of FIG. 5 represents a cross-sectional view taken as indicated. In this embodiment, sensor 18 includes multiple parallel strands of wire 19 formed around a frame 20. With multiple strands of wires, the resonant circuit may be created with either the inclusion of a discrete capacitor, element or by the inherent capacitance of the coils without the need for a separate capacitor as capacitance is provided between the wires 19 of the implant. Note that in the cross-sectional view of FIG. 5, individual ends of the very fine wires are not distinctly visible due to their small size. The wires are wrapped around frame 20 in such a way to give the appearance of layers in the drawing. Exact capacitance required for the RC circuit can be achieved by tuning of the capacitance through either or a combination of discrete capacitor selection and material selection and configuration of the wires. In one alternative sensor 18, there may be relatively few wire strands, e.g. in the range of about 15 strands, with a number of loops around the sensor in the range of about 20. In another alternative implant 18, there may be relatively more wire strands, e.g., in the range of 300 forming a single loop around the sensor.

The frame 20 may be formed from Nitinol, either as a shape set wire or laser cut shape. One advantage to a laser cut shape is that extra anchor features may cut along with the frame shape and collapse into the frame for delivery. When using a frame structure as shown in FIG. 5 the frame should be non-continuous so as to not complete an electrical loop within the implant. The coil wires may comprise fine, individually insulated wires wrapped to form a Litz wire. Factors determining inherent inductance include the number of strands and number of turns and balance of capacitance, frequency, Q, and profile.

Deriving Fluid Status from a Ratio of Cardiac to Respiratory Collapse

Increased blood volume can lead to hospitalisation and death. Pressure inside the vessel as well as geometric representations of the vessel size (i.e. volume, area, diameter) are typically used to estimate changes in fluid status. Yet, such absolute measures are strongly influenced by inaccurate and cumbersome measurement methods which hamper the ability to set unique thresholds in order to classify the fluid status in human. Furthermore, using a sensor touching the inside of a vessel of the human body can change the physiological response of the vessel itself. For example, sensor-vessel interaction may lead to tissue growth over a sensor. Such growth can decrease a given sensors ability to collapse for given vessel. Thus, the establishment of dimensional thresholds for a vessel would be affected and be required to change depending on the degree of sensor and vessel interaction.

A system 1 is provided for determining fluid status in a blood vessel 2. The system 1 comprises a sensor 4 configured to obtain a measurement from the vessel 2. A processor 5 is configured to derive a measure of cardiac collapse of the vessel from the measurement; derive a measure of respiratory collapse of the vessel from the measurement and furthermore to calculate a ratio of cardiac to respiratory collapse such that the calculated ratio provides an indication of the fluid status in the vessel.

Fluid status may thus be determined in a blood vessel from a ratio of cardiac to respiratory collapse observed from different types of measurement. For example, in time traces of pressure, or geometric measures such as volume, area, and diameter.

This provides for a simplified measurement technique when compared to other technologies used to obtain similar fluid status information—such as absolute blood volume measurements, external ultrasound and pulmonary implants of pressure sensors. Such techniques typically require the taking of a blood sample. Furthermore, measurements obtained may be noisy and prone to artefacts due to external factors. Also such techniques are typically “once off” and cannot be used for continuous monitoring, they merely provide a snapshot of patient's status at the time of testing.

The sensor configured to obtain the measurements from the vessel may be deployed in the blood vessel. For example, the sensor may be deployed in the interior vena cava (IVC). Once deployed, the sensor may be used to provide measurements as required. The need for repeated invasive measurements to be taken from a patient is obviated. The sensor may be that as shown in FIG. 4 or other sensor types may be used.

FIG. 5 shows a plot of absolute cross section of a sensor (in mm²) over a period of time. The relative changes in area due to both respiration and cardiac collapse are shown in aggregate. The sensor provides raw signal data which may be filtered to separate features associated with a cardiac response and features associated with a respiratory response in a patient. For example, a heart rate response will typically manifest itself as a signal displaying 50 to 100 bpm while a respiratory response will typically manifest itself as a signal displaying 2 to 50 bpm. A heart rate in a “dry case” may be hard to detect due to noise effects. It is possible to filter the respiratory signal and subtract from the raw signal data to thus leave a signal providing the cardiac output and noise. As noise is Gaussian, this may be further filtered to provide the cardiac output. As an alternative, it is possible to determine the magnitude and phase variation from an externally obtained sensor signal (for example, a belt type sensor with an accelerometer) to determine a respiration rate. This can be subtracted from raw signal data obtained from an internally deployed vessel sensor to obtain a cardiac signal.

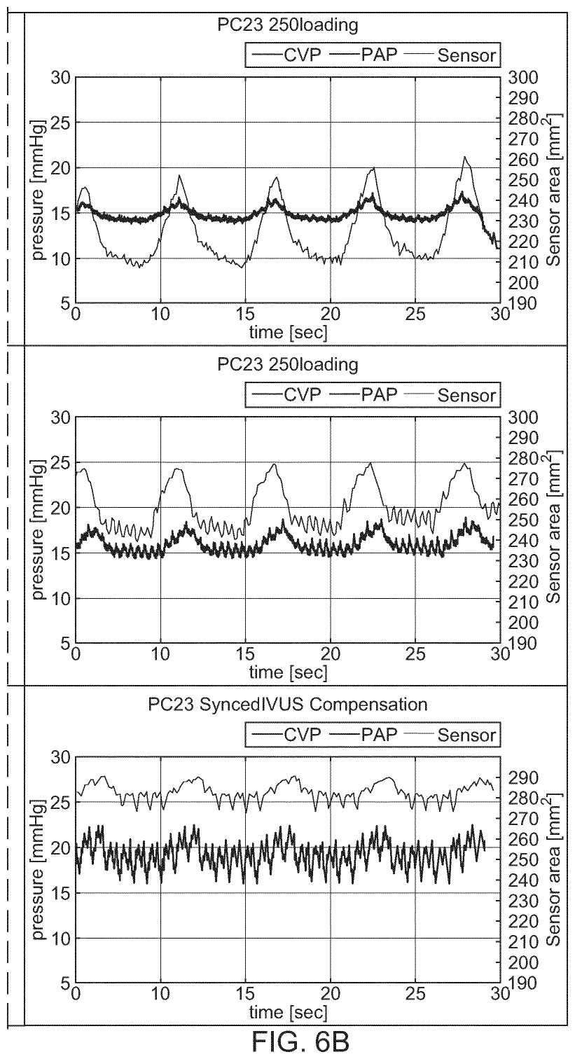

FIGS. 6A and 6B show sensor area and pressure measurement obtained from loading blood into healthy sheep with a weight of 70 kg. Measurements are obtained as fluid is loading and the condition changes from dry (low fluid load), normal and wet (high fluid load). These traces demonstrate the low cardiac to respiratory ratio in the dry case and the high cardiac to respiratory ratio in the wet case and this can be seen in both the pressure and area trace data.

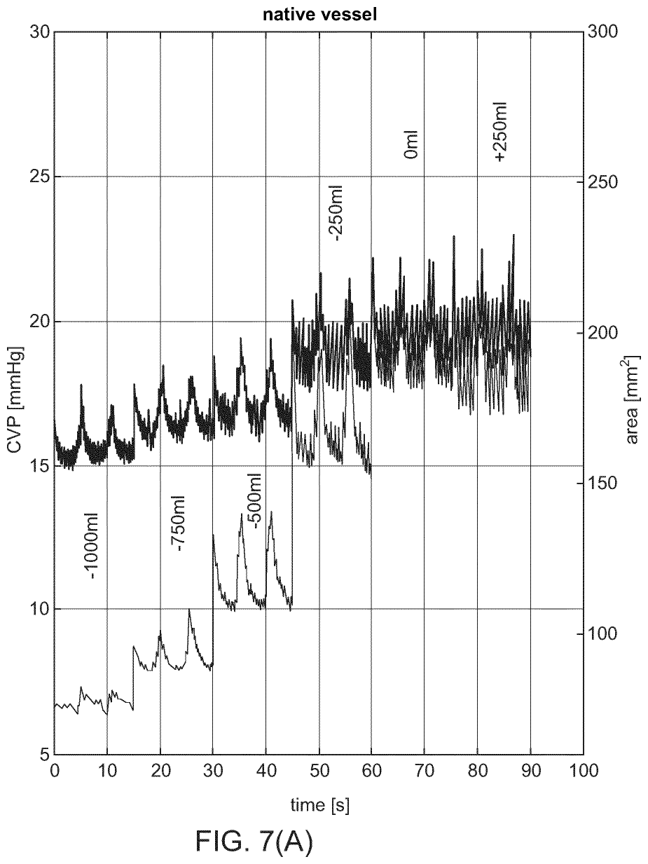

FIGS. 7A and 7B show IVC area and pressure changes in a native vessel (—top image labelled “native vessel”) and after deployment of a device (for example, the device of FIG. 4) into the IVC (lower image labelled “acute after sensor deployment”). Please note that the cardiac collapse becomes visible in both area and in pressure in native and acute conditions as a superimposed modulation on top of the respiration modulation from fluid levels of −500 ml blood volume added/withdrawn. A higher frequency cardiac pulse is only visible in the waveforms above −500 mls and is therefore an indication of fluid accumulation.

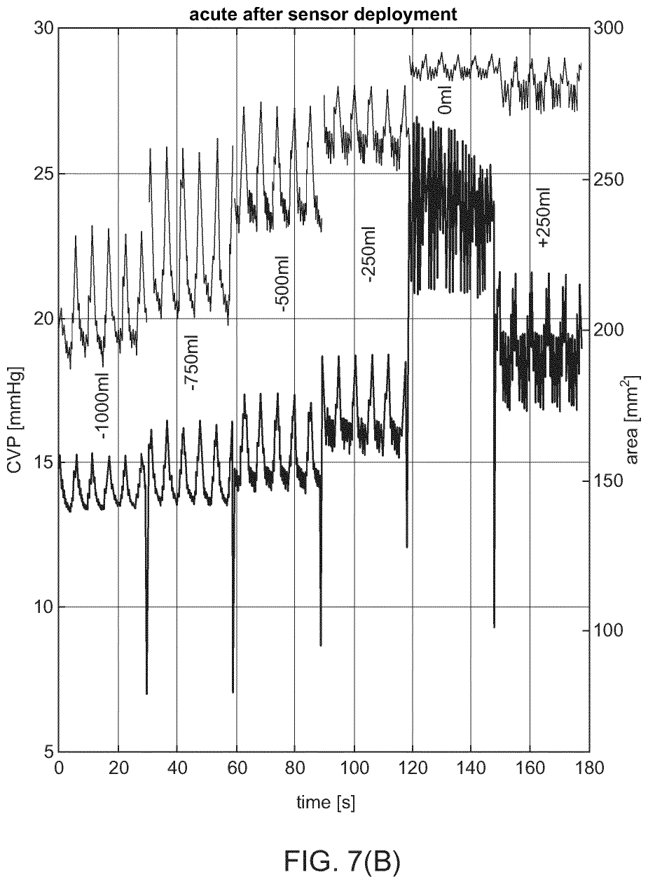

FIG. 8 shows further data obtained using the system described herein from further fluid loading testing in the IVC of sheep. The cardiac magnitude (% respiratory magnitude) is plotted against sensor area. The data points shown as shaded circles indicate the removal of blood in 250 ml steps. The data points shown as unshaded circles show the addition of blood in 250 ml steps. Once again this data demonstrates that the cardiac magnitude (% respiratory magnitude) increases with fluid loading and increasing area.

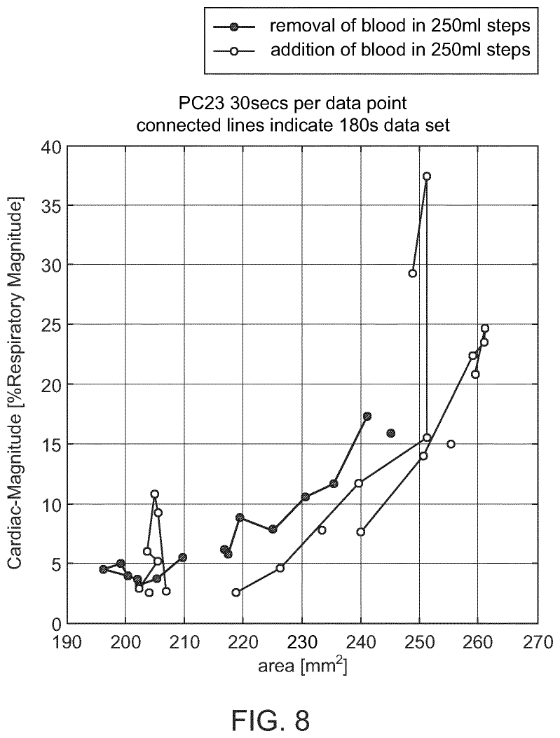

FIG. 9 shows data obtained using the system described herein from fluid loading testing in a native IVC of a heart failure patient. The graph shows the collapse ratio for baseline (far left), after 250 ml infusion of saline (centre) and after 500 ml infusion of saline (far right). The cardio-respiratory collapse ratio increases with amount of added fluid. The traces filtered from the raw signal are shown in the bottom left figure.

The system above is described with a sensor deployed in a vessel, for example the IVC, to obtain measurements from the vessel. It is estimated that such an arrangement provides for the precision of measurements obtained to be in the order of ten times higher than other modalities such as, for example, external ultrasound. The system described provides for precision in the region of +/−0.1 mm on the diameter of a vessel compared to external ultrasound, which provides for precision is in the region of +/−1 mm on the diameter of a vessel. Such enhanced accuracy provides for reliable determination of cardiac and respiratory collapse.

The measurement can a pressure measurement, the measurement may be in the form of an MRI image, the measurement may be a pulse oximetry measurement. The measurement may be a temporal trace recording, wherein the temporal trace recording is of vascular modulation or dimensional changes of the vessel. Furthermore, the sensor of the system may be applied to the skin of a patient, for example via a skin mounted patch.

A method is provided of determining fluid status in a blood vessel comprising obtaining a measurement from the vessel via a sensor; deriving a measure of cardiac collapse from the measurement; deriving a measure of respiratory collapse from the measurement; calculating a ratio of cardiac to respiratory collapse such that the calculated ratio provides an indication of the fluid status in the vessel.

Once a fluid status is obtained for a patient, the volume of fluid in the vessel may be adjusted based on the indication of the fluid status. As such, a patient's fluid status may be regulated based on the obtained fluid status measurements. The adjustment may take place by recommending a treatment schedule to include for example drug intake, dialysis, ultrafiltration, blood pumping. The obtained measurements can provide indications as to the most appropriate treatment schedule for a given patient.

Fluid Status Derived from Change in Radial Force

A system is provided for determining fluid status in a blood vessel, for example the IVC, comprising a resilient sensor, deployed in the blood vessel. The sensor (for example, the sensor of FIG. 4) can be configured to obtain a measurement from the vessel. The sensor is compressible between a maximally dimensioned size s1 (i.e. when the sensor is fully expanded), and a minimally dimensioned size s2 (i.e. when the sensor is fully compressed) (See for example, FIG. 12). A processor is configured to obtain a measurement, m1, of the change in sensor dimensions after deployment in the vessel, m1 being a value between and including s1 and s2. The processor is further configured to obtain from m1, a value of a radial force, r1 exerted by the sensor on the vessel after deployment in the vessel and furthermore to calculate a ratio of the change in a vessel dimension resulting from r1 after deployment of the sensor in the vessel with respect to a known vessel dimension, Ao, prior to deployment of the sensor in the vessel, wherein the ratio provides an indication of the fluid status in the vessel.

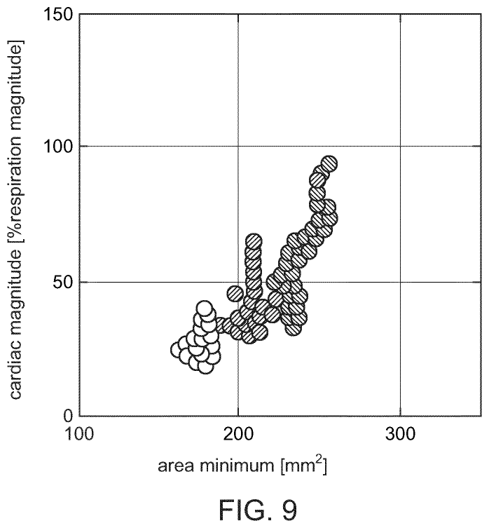

Further provided is a method for determining fluid status in a blood vessel comprising obtaining a vessel dimension, Ao, prior to deployment of a sensor in the vessel. The vessel dimensions may be obtained experimentally (for example, with reference to FIG. 10 below). Vessel dimensions may be obtained via ultrasound, X-Ray or MM imaging. The sensor is deployed in the vessel and is configured to obtain a measurement from the vessel. The sensor is compressible between a maximally dimensioned size s1, and a minimally dimensioned size s2. The method provides for obtaining a measurement, m1, of the change in sensor dimensions after deployment in the vessel, m1 being a value between and including s1 and s2; obtaining from m1, a value of a radial force, r1 exerted by the sensor on the vessel after deployment in the vessel and calculating a ratio of the change in a vessel dimension resulting from r1 after deployment of the sensor in the vessel, with respect to the obtained vessel dimension prior to deployment of the sensor in the vessel, wherein the ratio provides an indication of the fluid status in the vessel.

The sensor has known properties, e.g. tensile properties, minimum dimensions under compression, maximum dimensions upon extension, which may be calculated and calibrated prior to deployment in a blood vessel. The sensor thus exerts a known radial force onto the vessel wall upon deployment resulting from the compression or expansion of the sensor.

Fluid status is determined in a blood vessel using a known radial force and from a ratio of native to acute vessel maximum and minimum measurements observed in time traces of pressure, or geometric measures such as volume, area, and diameter.

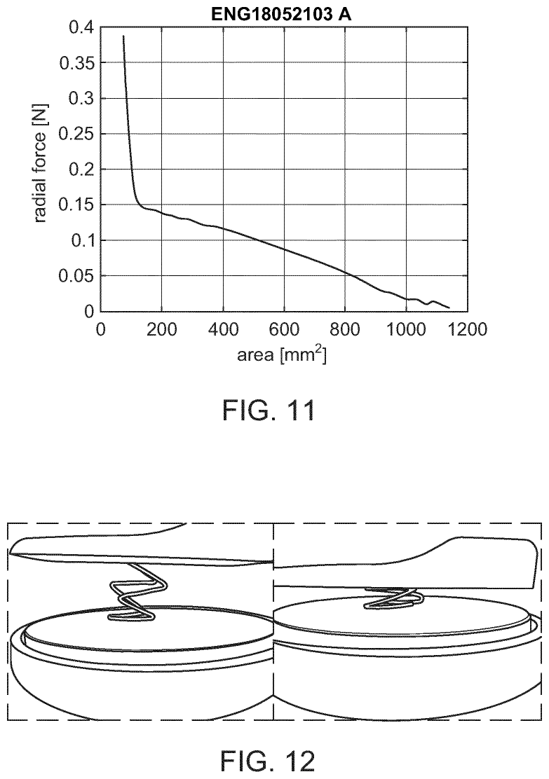

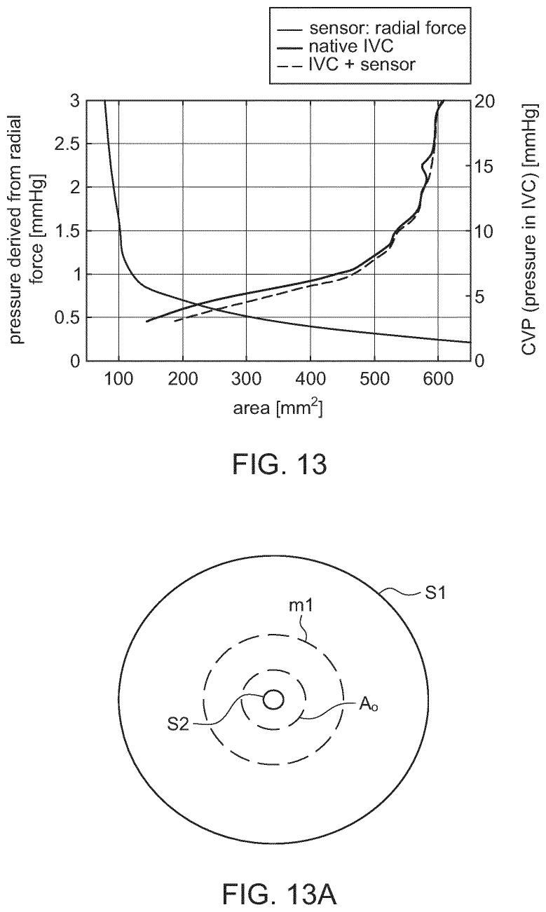

This system provides for determining a reference area for the IVC location that the device is implanted in using the radial force information obtained from the sensor. Without measuring pressure or driving the vessel through its full dynamic range geometrically it is challenging to know by how much the dimensions of a vessel can in fact still change. The present system makes use of a known and calibrated radial force added to the internal pressure keeping the vessel open. The change of the minimum and maximum vessel size due to the applied force can then be used to estimate whether the vessel can still expand or contract using an experimentally gained model of the pressure-volume curve of said vessel. In effect, it can be established where a given patients fluid status resides on the CVP-A curve, such as the example curve in FIG. 13. Thus, this provides an indication of a given patients fluid status at sensor implantation and can therefore be used as an input to understand future changes in sensor output.

In the present system, m1 provides a measurement of maximum change in sensor dimensions after deployment in the vessel, m1 being a value between and including s1 and s2. The processor is further configured to obtain a second measurement, m2, of minimum change sensor dimensions after deployment in the vessel, m2 being a value between and including s1 and s2. The processor calculates a ratio of m1 with respect to a known maximum vessel dimension (mₙₐₜᵢᵥₑ₁) to provide (m a MAXCHANGE value and a ratio of m2 with respect a known minimum vessel dimension (mₙₐₜᵢᵥₑ₂) to provide a MINCHANGE value.

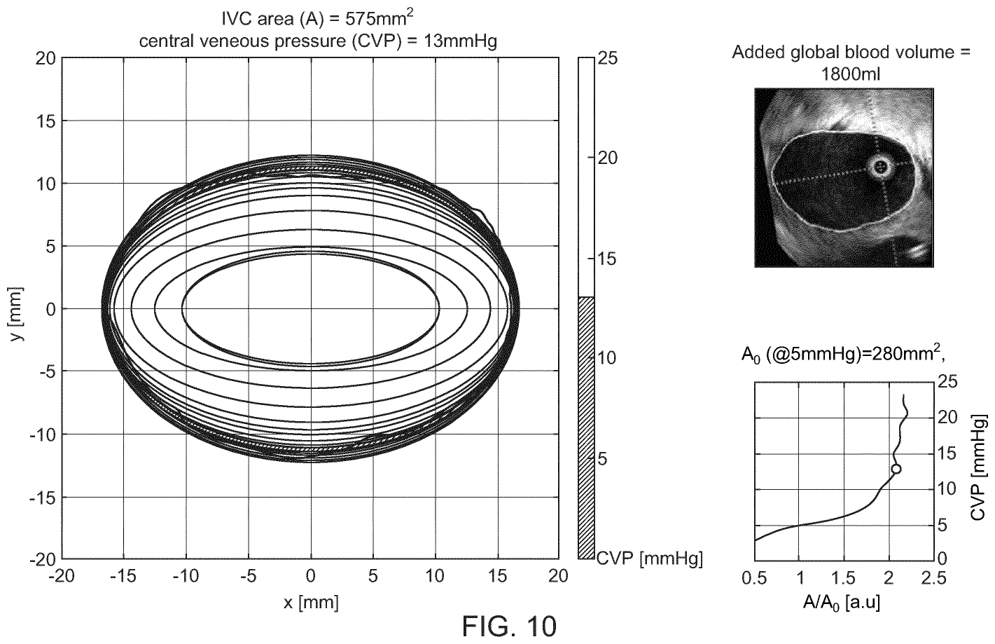

FIG. 10 shows measurements for determining a CVP-A/A0 curve experimentally for vein. An example of the curve is shown in the bottom right of the figure. FIGS. 11 and 12 show a calibration procedure to acquire radial force versus sensor area data for a sensor to be deployed. A sensor such as that shown in FIG. 4 is under test, however other sensor types may be used. The sensor is subjected to a series of test forces. In this manner, s1 and s2 of a given sensor can be obtained as well as the radial force exerted by the sensor across its full range of compressed and expanded positions. FIG. 13 shows adjusted model merging radial force of sensor curve and experimentally obtained native vessel curve for pressure and volume/area. These calibrated figures can be used in correlation which the measured compression and expansion of the sensor upon inspiration and expiration of a patient to determine the radial force exerted by the sensor in the vessel. These values can be used to obtain an indication of the fluid status of the patient. FIG. 13A shows a schematic of maximum and minimum sensor sizes s1, s2 as described above in a vessel along with an example measurement m1. In this example, the vessel diameter Ao, prior to deployment is expanded to a size m1. An increase in the value of m1 corresponds to a decrease in the radial force exerted by the sensor.

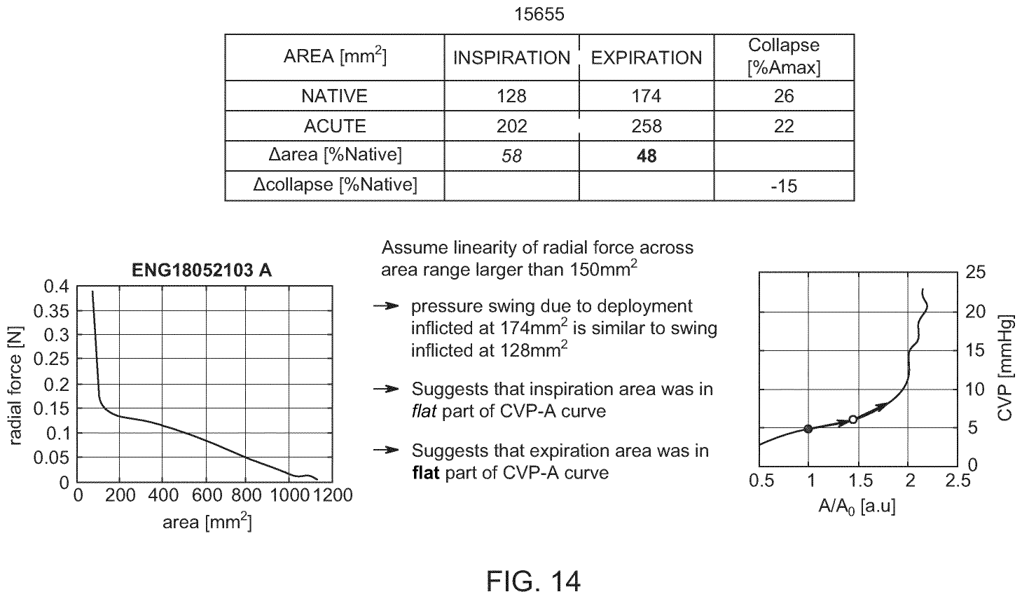

For example, with reference to FIG. 14 is it shown that the area change due to deployment of the sensor inflicted at 174 mm² is similar to the change inflicted at 128 mm². Inspiration values correspond to MINCHANGE values while expiration values correspond to MAXCHANGE values. This suggests that the inspiration area was in the flat part of the CVP-A curve. This further suggests that the expiration area was in the flat part of the CVP-A curve. This indicates a fluid status of “close to normal”.

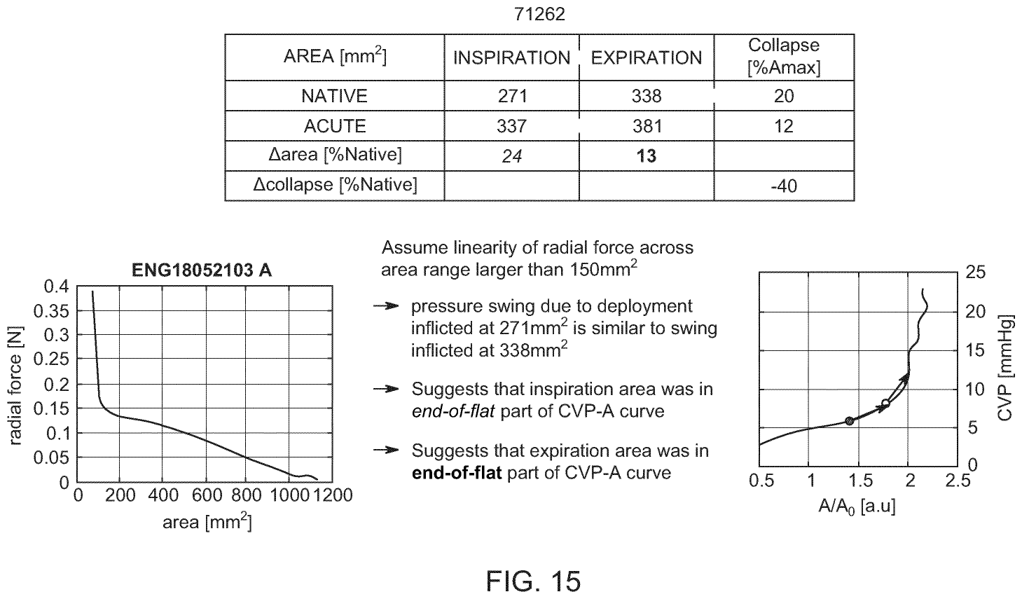

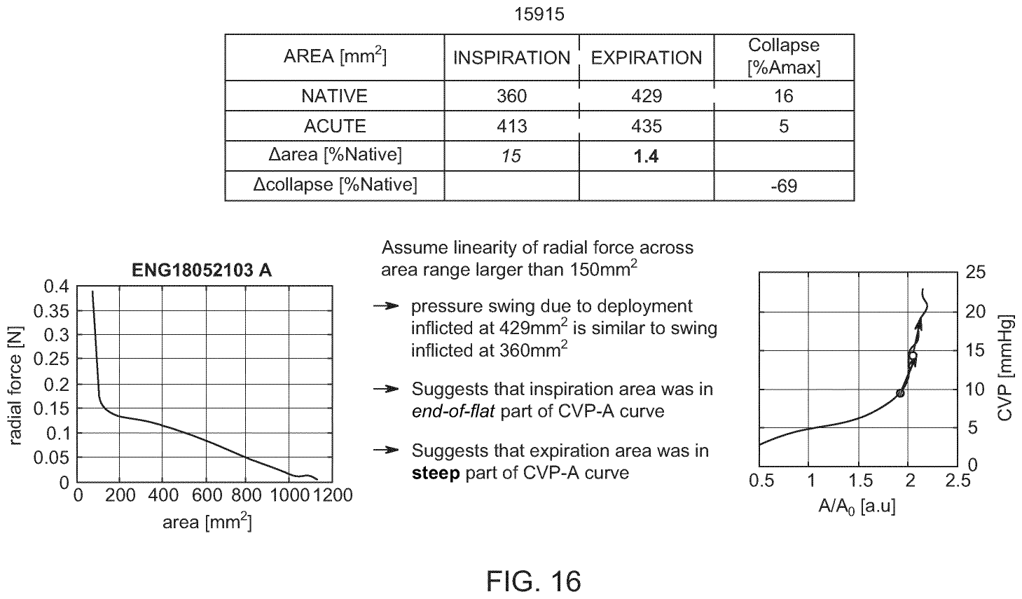

With reference to FIG. 15 is it shown that the area change due to deployment of the sensor inflicted at 271 mm² is twice the change inflicted at 338 mm². This suggests that the inspiration area was in the end of the flat part of the CVP-A curve. This further suggests that the expiration area was at the beginning of the steep part of the CVP-A curve. This indicates a fluid status of “normal to moderately full”. With reference to FIG. 16 is it shown that the area change due to deployment of the sensor inflicted at 429 mm² is 10 times smaller compared to the change inflicted at 360 mm². This suggests that the inspiration area was in the end of the flat part of the CVP-A curve. This further suggests that the expiration area was in the steep of the flat part of the CVP-A curve. This indicates a fluid status of “full”.

Thus these figures allow to provide guidelines for assessing a position on a P-V curve by assessing the change in area for a native vessel to a vessel with a sensor deployed. This is summarized in Table 1 below.

TABLE 1

| | | Inspiration | Expiration |
|---|---|---|---|
| | | (corresponding to | (corresponding to |
| P-V Curve | | MINCHANGE | MAXCHANGE |
| Location | Fluid status | value) | value) |
| Flat Part | Normal | Large Change | Large Change |
| | | (>20%) | (>20%) |
| Intermediate | Normal to | Large Change | Small Change |
| | Moderately Full | (>20%) | (<20% & >10%) |
| Steep | Full | Small Change | Very Small Change |
| | | (<20% & >10%) | (<10%) |

A large change may be considered to be of the order of a greater than 20% area change, while a small change may be considered to be of the order of a less than 10% area change.

Thus, a full fluid status is indicated by a MAXCHANGE value being less than a MINCHANGE value by a factor of F1, wherein F1 is about 10. A normal to moderately full fluid status is indicated by a MAXCHANGE value being less than a MINCHANGE value by a factor of F2, wherein F2 is about 2. A normal status is indicated by a MAXCHANGE value being less than a MINCHANGE value by a factor of F3, wherein F3 is about 1.2 to 1.5.

Method for Detecting Congestion

There are a number of existing techniques for detecting congestion in a patient, for example blood pressure and heart rate monitoring, jugular venous distension, point of maxima impulse measurements, 3rd and 4th heart sounds detection, pulmonary exam, liver size examination and hepatojugular reflux and lower extremity edema. These techniques all suffer from the disadvantage that they must be performed by a skilled technician in a clinic.

Measurement of blood pressure during a Valsalva manoeuvre has been described in relation to cardiac congestion assessment as early as 1976 by Wilkinson et al. This method does however require invasive pressure measurements to be obtained and is therefore not appropriate for home use.

A system is provided for determining congestion in a blood vessel comprising a sensor in the vessel. The sensor is configured to obtain a first area measurement, a1, of the vessel prior to patient manoeuvre and a second area measurement, a2, of the vessel after a patient manoeuvre.

A processor configured to determine the congestion in a blood vessel of the vessel based on the first and second area measurements. The sensor may be a sensor as shown in FIG. 4 although other sensor types may be used. The sensor may be deployed in the IVC of a patient.

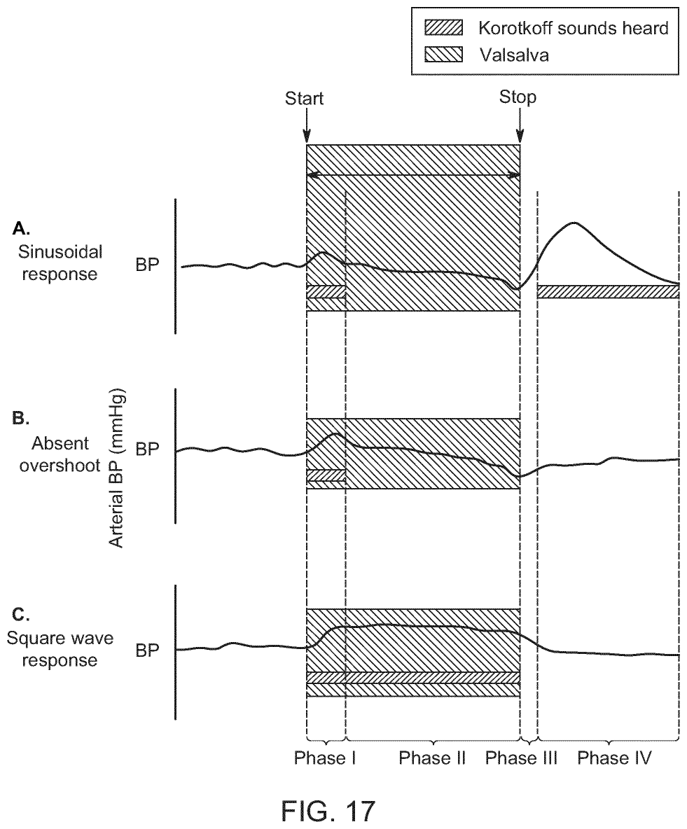

The processor is configured to provide a signal output based on the area measurements taken prior to and after the patient manoeuvre. The processor is further configured to determine the fluid status in the blood vessel based on an identified signal shape derived from the first and second area measurements. When the patient manoeuvre is a Valsalva manoeuvre, the identified signal shape is a square wave shape. Effectively, the system provides for evaluating the IVC response to the manoeuvre. If a square wave pattern in the IVC dimensions is observed in the signal, this provides an indication that the patient is fluid overloaded, in the same manner as has been described previously using blood pressure as the input signal (See FIG. 17).

The processor is further configured to provide a notification of detected congestion in the blood vessel. This notification can be in the form of a computer readout. Alternatively, the notification may be transmitted to a remote monitoring server or may be transmitted to a wireless handheld device. The processor is further configured to provide a notification indicating an action for reducing congestion in the blood vessel.

For example, the action can comprises a drug treatment change, a medical treatment schedule. The medical treatment schedule may comprise at least of a diuretic or vasodilation schedule, a modification to a medical device such as vascular pump, drug pump, dialysis or auto-filtration machine, pacing device or extracorporeal membrane oxygenation (ECMO) machine.

As such, when a sensor is implanted in the manner described, daily measurement of congestion in the home by patients is feasible. This provides for earlier detection and for tailored treatment schedule specific to a patient's needs.

A method for determining congestion in a blood vessel is as follows:
a first area measurement, a1, is obtained from a sensor in a blood vessel prior to performing a patient manoeuvre;
the patient manoeuvre is performed, for example a Valsalva manoeuvre;
a second area measurement, a2, is obtained from the sensor in the blood vessel after performing the patient manoeuvre;
the congestion in the blood vessel is obtained based on the first and second area measurements.

The first and second measurements provide a signal output. Detection of a square wave pattern in the signal output provides an indication that the patient is fluid overloaded.

Assessing Cardiac Output by Monitoring IVC Area Changes

Change in cardiac output is a key indicator for patient with heart failure. Being able to monitor cardiac output remotely allows optimum care of patients with heart failure, enabling physicians to improve quality of life and life expectancy for such patients.

Cardiac output is typically determined through performing an angiogram. However, this requires a hospital visit and is invasive.

A system for determining cardiac output, O_{c} is provided comprising: a sensor deployed in the inferior vena cava, IVC, the sensor configured to obtain a first area measurement, Area1, of the IVC at a time t1. The sensor is further configured to obtain a second area measurement, Area2, of the IVC at a time t2. A processor is configured to determine the cardiac output based on an area change of the IVC derived from the first and second area measurements.

The processor is configured to determine the cardiac output from the obtained area measurements as the cardiac output O_{c} is proportional to changes in the area of the IVC.

The processor is further configured to provide a notification of the cardiac output. This provides an automatic presentation of information regarding cardiac output without the need for further analysis or computation.

This notification can be in the form of a computer readout. Alternatively, the notification may be transmitted to a remote monitoring server or may be transmitted to a wireless handheld device. The processor is further configured to provide a notification indicating an action for adjusting cardiac output in the blood vessel.

For example, the action can comprises a drug treatment schedule, a medical treatment schedule. The medical treatment schedule can include dialysis schedule, treatment Y, treatment Z. As such, when a sensor is implanted in the manner described daily measurement of cardiac output in the home by patients is feasible. This provides for earlier detection and for tailored treatment schedule specific to a patient's needs.

Further provided is a method for determining cardiac output comprising: obtaining a first area measurement, Area1, from a sensor deployed in the inferior vena cava, IVC, at a time t1; obtaining a second area measurement, Area2, from a sensor deployed in the IVC, at a time t2; and determining the cardiac output based on an area change of the IVC derived from the first and second area measurements.

Monitoring area changes of IVC can thus be an indicator of cardiac output Cₒ of a patient. Sensors may be deployed in a patient as described above in order to obtain area measurements of the IVC

Deriving Cardiac Output O_{c} from IVC Area Measurements

Changes in the area of the IVC may be used to derive an indication of cardiac output.

Change in cardiac output Cₒ is linked to changes in Venous Return. Venous return may be determined from a combination of IVC flow and SVC flow wherein SVC is the Superior Vena Cava. A factor IVC_{flow milking} is derived from a sum of volume changes of IVC wrt time. This assumes Volume changes of IVC are dominated by area changes of the IVC. Furthermore, it is assumed that change in pressure on the respiration cycle is dominating the pressure drive of volume change related to IVC_{flow milking}.

IVC_{flow milking} directly correlates to cardiac output therefore area changes of the IVC can be an indicator of cardiac output O_{c}.

Venous Resistance

Furthermore Venous Resistance can be measured. Firstly, Venous Return may is defined by

$$Venous Return = \frac{RAP - MCFP}{Ven Resistance}$$

where MCFP=Mean Circulatory Filling pressure
RAP=Right Atrial Pressure

Therefore:

$$VenResistance = \frac{RAP - MCFP}{VenousR eturn}$$

It is assumed that changes in Venous flow are dominated by a flow factor—Volumemilking. It is assumed that change in pressure on the respiration cycle is dominating the pressure drive of Volumemilking. It is assumed that Venresistance does not change over the respiration cycle

For the respiration cycle—Volumemilking is correlated to the volume of the IVC

At minimum IVC pressure during breathing—Flow of Volumemilking=0, while at maximum IVC pressure—Flow of Volumemilking is maximum.

Venous Resistance (VenResistance) may be defined by:

$$VenResistance = \frac{Δ P milking}{Volumemilking}$$

Where ΔPmilking may be derived from pressure changes in the IVC determined from area changes of the IVC.

Note changes of Venous flow if RAP increases or MCFP decreases; MCFP is when Venous Flow is zero. If milking flow is close to zero at low pressure, the pressure at smallest related will be related to MCFP.

The words “comprises/comprising” and the words “having/including” when used herein with reference to the present disclosure are used to specify the presence of stated features, integers, steps or components but do not preclude the presence or addition of one or more other features, integers, steps, components or groups thereof.

It is appreciated that certain features of the disclosure, which are, for clarity, described in the context of separate embodiments, may also be provided in combination in a single embodiment. Conversely, various features of the disclosure which are, for brevity, described in the context of a single embodiment, may also be provided separately or in any suitable sub-combination.

1.-24. (canceled)

25. A system for determining congestion in a blood vessel comprising:
a sensor configured to be disposed in the vessel, the sensor further configured to obtain a first signal indicating a first area measurement, a1, of the vessel prior to patient maneuver and a second signal indicating a second area measurement, a2, of the vessel after a patient maneuver;
a processor configured to determine the congestion in a blood vessel of the vessel based on the first and second signals.

26. The system of claim 25, wherein the processor is further configured to determine a patient fluid status based on an identified signal shape derived from the first and second area measurement signals.

27. The system of claim 26, wherein the identified signal shape is a square wave shape.

28. The system of claim 25, wherein the processor is further configured to provide a notification of a patient fluid status based on the first and second signals.

29. The system of claim 25, wherein the processor is further configured to provide a notification indicating an action for reducing congestion in the blood vessel.

30. The system of claim 29, wherein the action comprises a drug treatment schedule.

31. The system of claim 29, wherein the action comprises a medical treatment schedule.

32. The system of claim 31, wherein the medical treatment schedule comprises at least of a diuretic or vasodilation schedule, a modification to a medical device such as vascular pump, drug pump, dialysis or auto-filtration machine, pacing device or extracorporeal membrane oxygenation (ECMO) machine.

33. A method for determining congestion in a blood vessel comprising:
obtaining a first signal indicating a first area measurement, a1, from a sensor in a blood vessel prior to a patient performing a patient maneuver;
prompting the patient to perform the maneuver;
obtaining a second signal indicating a second area measurement, a2, from the sensor in the blood vessel after the patient maneuver;
determining the congestion in a blood vessel of the vessel based on the first and second signals.

34. The method of claim 33, further comprising placing the sensor in the blood vessel and wherein the patient maneuver is a Valsalva maneuver.

35. A system for determining cardiac output, O_{c}, comprising:
a sensor deployed in the inferior vena cava, IVC, the sensor configured to obtain a first area measurement, Area1, of the IVC at a time t1;
the sensor configured to obtain a second area measurement, Area2, of the IVC at a time t2; and
a processor configured to determine the cardiac output based on area changes of the IVC derived from the first and second area measurements.

36. The system of claim 35, wherein the processor is further configured to determine a heart rate from analysis of the area changes of the IVC.

37. The system of claim 35, wherein the processor is further configured to provide a notification of the cardiac output.

38. The system of claim 37, wherein the processor is further configured to provide a notification indicating an action for adjusting the cardiac output.

39. The system of claim 38, wherein the action comprises a drug treatment schedule.

40. The system of claim 38, wherein the action comprises a medical treatment schedule.

41. The system of claim 40, wherein the medical treatment schedule comprises at least of a diuretic or vasodilation schedule, a modification to a medical device such as vascular pump, drug pump, dialysis or auto-filtration machine, pacing device or extracorporeal membrane oxygenation (ECMO) machine.

42. A method for determining cardiac output, comprising:
obtaining a first area measurement, m1, from a sensor deployed in the inferior vena cava, IVC, at a time t1;
obtaining a second area measurement, m2, from a sensor deployed in the IVC, at a time t2; and
determining the cardiac output based on an area change of the IVC derived from the first and second area measurements.
